(19) United States
(12) Patent Application Publication
(10) Pub. No.: US 2025/0297032 A1
(43) Pub. Date: Sep. 25, 2025

(54) HUMAN ANTIBODIES AGAINST FAP-ALPHA

(71) Applicants: CONCEPT TO MEDICINE BIOTECH CO., LTD., Shanghai (CN); LEPU BIOPHARMA CO., LTD., Shanghai (CN)

(72) Inventors: Liu YANG, Shanghai (CN); Wenqing JIANG, Shanghai (CN); Lei FANG, Shanghai (CN)

(21) Appl. No.: 18/724,173

(22) PCT Filed: Dec. 29, 2022
(86) PCT No.: PCT/CN2022/143358

§ 371 Date: Jun. 25, 2024

(30) Foreign Application Priority Data
Dec. 30, 2021 (WO) PCT/CN2021/143087

(51) Int. Cl. C07K 16/40; A61K 40/31; A61K 40/42; C07K 14/725

(52) U.S. Cl. CPC C07K 16/40; A61K 40/31; A61K 40/4249; C07K 14/7051; A61K 2239/13; A61K 2239/21; A61K 2239/22; C07K 2317/31; C07K 2317/33; C07K 2317/565; C07K 2317/92

(57) ABSTRACT

Provide are full human anti-FAPα antibodies and variants thereof with further optimized CDR sequences. The new antibodies exhibited high affinity to the FAPα protein and can be used to treat cancers and inflammatory conditions.

### CROSS REFERENCE TO RELATED APPLICATIONS

The present application is a U.S. National Stage of PCT International Patent Application No. PCT/CN2022/143358 filed Dec. 29, 2022, which claims priority of PCT International Patent Application No. PCT/CN2021/143087 filed Dec. 30, 2021, each of which is incorporated herein by reference in its entirety.

### REFERENCE TO AN ELECTRONIC SEQUENCE LISTING

The contents of the electronic sequence listing “347044.xml” size 64,529 bytes, and Date of Creation Jun. 12, 2024, is herein incorporated by reference in its entirety.

### BACKGROUND

FAPα (fibroblast activation protein alpha, FAP, FAPA, or FAPalpha) is an integral membrane gelatinase, which is over-expressed in cancer-associated fibroblasts (CAFs). CAFs are tumor stromal cells that represents the most prominent components of tumor microenvironments. CAFs modulate multiple aspects of tumor growth, including tumorigenesis, angiogenesis, metastasis, immunosuppression and promotion of drug resistant. FAPα is thought to be involved in the control of fibroblast growth or epithelial-mesenchymal interactions during development, tissue repair, and epithelial carcinogenesis.

FAPα is selectively expressed in reactive stromal fibroblasts of epithelial cancers, granulation tissue of healing wounds, and malignant cells of bone and soft tissue sarcomas. Across a wide range of human cancer types, such as gastric carcinoma, pancreatic cancer, breast cancer and colon cancer, the expression of FAPα has been reported to correlate with a higher tumor grade and worse overall survival in solid tumors. At present, FAP inhibitors have been widely developed in tumor PET/computed tomography (CT) imaging. Theoretically, FAP-targeted drugs could also serve as a promising therapeutic target for inhibition of tumor progression and metastasis. Nonetheless, no satisfactory therapeutic effect has been observed so far. Therefore, considering the important role of FAPα in tumor progression and its rare expression in healthy tissues, it will be worthwhile to further investigate the therapeutic value of FAPα in treatment of cancer.

In addition, it was observed that high expression of FAPα in RA (rheumatoid arthritis) fibroblast-like synoviocytes (FLSs) is associated with an invasive phenotype of FLSs accompanied by high proliferation and destruction of the extracellular matrix. Therefore, FAPα can be considered as a therapeutic target to inhibit the destructive potential of FLSs.

### SUMMARY

The present disclosure, in various embodiments, provides full human antibodies and antigen-binding fragments specific to the human FAPα protein. Experimental testing shows that these newly identified antibodies can bind to the human FAPα protein potently and specifically. In addition, the majority of them retain the potent affinity to the FAPα protein expressed on cell surfaces, and can be suitably used for treating diseases associated with FAPα expression.

In accordance with one embodiment of the present disclosure, provided is an antibody or antigen-binding fragment thereof which has specificity to the human fibroblast activation protein alpha (FAPα) protein and comprises a heavy chain variable region (VH) comprising a VH CDR1, a VH CDR2 and a VH CDR3, and a light chain variable region (VL) comprising a VL CDR1, a VL CDR2, and a VL CDR3.

In some embodiments, the VH CDR1 comprises the amino acid sequence of SEQ ID NO: 67; the VH CDR2 comprises an amino acid sequence selected from the group consisting of SEQ ID NO: 15-17 and 43-55; the VH CDR3 comprises an amino acid sequence selected from the group consisting of SEQ ID NO: 20-23, 25, 59 and 60; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69.

In some embodiments, the VH CDR1 comprises the amino acid sequence of SEQ ID NO: 67; the VH CDR2 comprises an amino acid sequence selected from the group consisting of SEQ ID NO: 15 and 43-48; the VH CDR3 comprises an amino acid sequence selected from the group consisting of SEQ ID NO: 20, 59 and 60; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69. In some embodiments, the VH CDR1 comprises the amino acid sequence of SEQ ID NO: 11; the VH CDR2 comprises an amino acid sequence selected from the group consisting of SEQ ID NO: 15 and 43-48; the VH CDR3 comprises an amino acid sequence selected from the group consisting of SEQ ID NO: 20, 59 and 60; the VL CDR1 comprises the amino acid sequence of SEQ ID NO: 27; the VL CDR2 comprises the amino acid sequence of SEQ ID NO: 31; and the VL CDR3 comprises an amino acid sequence selected from the group consisting of SEQ ID NO: 34, 61 and 62.

In some embodiments, the VH CDR1 comprises the amino acid sequence of SEQ ID NO: 67; the VH CDR2 comprises an amino acid sequence selected from the group consisting of SEQ ID NO: 16 and 49-54; the VH CDR3 comprises the amino acid sequence of SEQ ID NO: 21; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69. In some embodiments, the VH CDR1 comprises the amino acid sequence of SEQ ID NO: 12; the VH CDR2 comprises an amino acid sequence selected from the group consisting of SEQ ID NO: 16 and 49-54; the VH CDR3 comprises the amino acid sequence of SEQ ID NO: 21; the VL CDR1 comprises the amino acid sequence of SEQ ID NO: 28; the VL CDR2 comprises the amino acid sequence of SEQ ID NO: 31; and the VL CDR3 comprises an amino acid sequence selected from the group consisting of SEQ ID NO: 35, 63 and 64.

In some embodiments, the VH CDR1 comprises the amino acid sequence of SEQ ID NO: 67; the VH CDR2 comprises the amino acid sequence of SEQ ID NO: 17 or 55; the VH CDR3 comprises the amino acid sequence of SEQ ID NO: 22; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69. In some embodiments, the VH CDR1 comprises the amino acid sequence of SEQ ID NO: 12; the VH CDR2 comprises the amino acid sequence of SEQ ID NO: 17 or 55; the VH CDR3 comprises the amino acid sequence of SEQ ID NO: 22; the VL CDR1 comprises the amino acid sequence of SEQ ID NO: 27; the VL CDR2 comprises the amino acid sequence of SEQ ID NO: 31; and the VL CDR3 comprises an amino acid sequence selected from the group consisting of SEQ ID NO: 34, 61 and 62.

In some embodiments, the VH CDR1 comprises the amino acid sequence of SEQ ID NO: 67; the VH CDR2 comprises the amino acid sequence of SEQ ID NO: 17 or 55; the VH CDR3 comprises the amino acid sequence of SEQ ID NO: 23; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69.

In some embodiments, the VH CDR1 comprises the amino acid sequence of SEQ ID NO: 12; the VH CDR2 comprises the amino acid sequence of SEQ ID NO: 17 or 55; the VH CDR3 comprises the amino acid sequence of SEQ ID NO: 23; the VL CDR1 comprises the amino acid sequence of SEQ ID NO: 27; the VL CDR2 comprises the amino acid sequence of SEQ ID NO: 31; and the VL CDR3 comprises an amino acid sequence selected from the group consisting of SEQ ID NO: 36, 65 and 66.

In some embodiments, the VH CDR1 comprises the amino acid sequence of SEQ ID NO: 67; the VH CDR2 comprises the amino acid sequence of SEQ ID NO: 17 or 55; the VH CDR3 comprises the amino acid sequence of SEQ ID NO: 25; the VL CDR1 comprises the amino acid sequence of SEQ ID NO: 68; the VL CDR2 comprises the amino acid sequence of SEQ ID NO: 31; and the VL CDR3 comprises the amino acid sequence of SEQ ID NO: 69. In some embodiments, the VH CDR1 comprises the amino acid sequence of SEQ ID NO: 12; the VH CDR2 comprises the amino acid sequence of SEQ ID NO: 17 or 55; the VH CDR3 comprises the amino acid sequence of SEQ ID NO: 25; the VL CDR1 comprises the amino acid sequence of SEQ ID NO: 27; the VL CDR2 comprises the amino acid sequence of SEQ ID NO: 31; and the VL CDR3 comprises an amino acid sequence selected from the group consisting of SEQ ID NO: 34, 61 and 62.

Also provided are multispecific antibodies comprising an antigen-binding fragment of the present disclosure and one or more antibody or antigen-binding fragment having binding specificity to a target antigen that is not FAPα.

Also provided, in another embodiment, is a chimeric antigen receptor (CAR) comprising an antigen-binding fragment of the present disclosure, a transmembrane domain, a costimulatory domain, and a CD3ξ intracellular domain.

Polynucleotides are also provided, encoding the antibody or antigen-binding fragment thereof or the CAR of the present disclosure. In some embodiments, the polynucleotide is mRNA, which is optionally chemically modified.

Methods and uses for treating cancer and inflammatory conditions are also provided, with the antibody or antigen-binding fragment thereof of the present disclosure.

### BRIEF DESCRIPTION OF THE DRAWINGS

FIG. 1 shows the ELISA binding activity of all tested antibodies with human and mouse FAPα protein.

FIG. 2 shows that, except I38 and J59, the tested antibodies bound to the FAPα protein on CHO-K1-hFAPα cells with high affinity.

FIG. 3 shows the Biacore kinetic testing results for each tested antibody.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes aptamers, spiegeleisen, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively.

The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

### Anti-FAPα Antibodies

As demonstrated in the appended experimental examples, the instant inventors were able to generate full human anti-FAPα antibodies G14, G52, I30, I37, I38, J40 and J59 (Table 1) all of which have high binding affinity to the human FAPα protein. Moreover, at least five of them, G14, G52, I30, I37 and J40, retained the superior affinity to cell-surface FAPα, making them suitable molecules for therapeutic applications. Further experiments demonstrate that all of antibodies target the same epitope on FAPα as a benchmark anti-FAPα antibody. Interestingly, these antibodies share highly homologous VH CDR1 and all VL CDR sequences.

In accordance with one embodiment of the present disclosure, provided is an antibody or antigen-binding fragment thereof. In some embodiments, the antibody or antigen-binding fragment thereof has binding specificity to the human FAPα protein. In some embodiments, the antibody or antigen-binding fragment thereof includes a heavy chain variable region (VH) that includes a VH CDR1, a VH CDR2 and a VH CDR3, and a light chain variable region (VL) that includes a VL CDR1, a VL CDR2, and a VL CDR3.

In some embodiments, the VH CDR1 includes the amino acid sequence of SEQ ID NO: 67, the VH CDR2 includes an amino acid sequence selected from the group consisting of SEQ ID NO: 15-17 and 39-47, the VH CDR3 includes an amino acid sequence selected from the group consisting of SEQ ID NO: 20-23, 25 and 51, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

As shown in Table 1C, SEQ ID NO: 67 has the sequence of SYAMX, where X is H or S. SEQ ID NO: 68 has the sequence of RASQGX₁X₂SWLA, where X₁ is I or V, and X₂ is Gor S. SEQ ID NO: 69 has the sequence of QQAX₁X₂FPX₃T, where X₁ is N or W, X₂ is A or S, and X₃ is L, P or V.

In some embodiments, provided is an antibody or antigen-binding fragment that is derived from antibody G14. In some embodiments, the VH CDR1 includes the amino acid sequence of SEQ ID NO: 67, the VH CDR2 includes an amino acid sequence selected from the group consisting of SEQ ID NO: 15 and 43-48, the VH CDR3 includes an amino acid sequence selected from the group consisting of SEQ ID NO: 20 and 51, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

In some embodiments, the VH CDR1 includes the amino acid sequence of SEQ ID NO: 11, the VH CDR2 includes the amino acid sequence of SEQ ID NO: 15, the VH CDR3 includes the amino acid sequence selected from the group consisting of SEQ ID NO: 20, the VL CDR 1 includes the amino acid sequence of SEQ ID NO: 27, the VL CDR2 includes the amino acid sequence of SEQ ID NO: 31, and the VL CDR3 includes the amino acid sequence of SEQ ID NO: 34.

In some embodiments, one or more of the CDRs is PTM (post-translational modification) de-risked. In some embodiments, the VH CDR1 includes the amino acid sequence of SEQ ID NO: 11, the VH CDR2 includes an amino acid sequence selected from the group consisting of SEQ ID NO: 15 and 43-48, the VH CDR3 includes the amino acid sequence selected from the group consisting of SEQ ID NO: 20, 59 and 60, the VL CDR1 includes the amino acid sequence of SEQ ID NO: 27, the VL CDR2 includes the amino acid sequence of SEQ ID NO: 31, and the VL CDR3 includes an amino acid sequence selected from the group consisting of SEQ ID NO: 34, 61 and 62.

In some embodiments, the VH includes the amino acid sequence of SEQ ID NO: 1 or a sequence having at least 75%, 80%, 85%, 90%, 95% or 99% sequence identity to SEQ ID NO: 1, while retaining the VH CDRs of SEQ ID NO: 1 or PTM re-risked versions thereof. In some embodiments, the VL includes the amino acid sequence of SEQ ID NO: 2 or a sequence having at least 75%, 80%, 85%, 90%, 95% or 99% sequence identity to SEQ ID NO: 2, while retaining the VL CDRs of SEQ ID NO: 2 or PTM re-risked versions thereof. In some embodiments, the VH includes the amino acid sequence of SEQ ID NO: 1, and the VL includes the amino acid sequence of SEQ ID NO: 2.

Also provided, in some embodiments, are antibodies and antigen-binding fragments therefore that bind to the same epitope on FAPα as G14. Also provided, in some embodiments, are antibodies and antigen-binding fragments therefore that competes with G14 in binding to FAPα.

In some embodiments, provided is an antibody or antigen-binding fragment that is derived from antibody G52. In some embodiments, the VH CDR1 includes the amino acid sequence of SEQ ID NO: 67, the VH CDR2 includes an amino acid sequence selected from the group consisting of SEQ ID NO: 16 and 49-54, the VH CDR3 includes the amino acid sequence of SEQ ID NO: 21, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

In some embodiments, the VH CDR1 includes the amino acid sequence of SEQ ID NO: 12, the VH CDR2 includes the amino acid sequence of SEQ ID NO: 16, the VH CDR3 includes the amino acid sequence selected from the group consisting of SEQ ID NO: 21, the VL CDR1 includes the amino acid sequence of SEQ ID NO: 28, the VL CDR2 includes the amino acid sequence of SEQ ID NO: 31, and the VL CDR3 includes the amino acid sequence of SEQ ID NO: 35.

In some embodiments, one or more of the CDRs is PTM (post-translational modification) de-risked. In some embodiments, the VH CDR1 includes the amino acid sequence of SEQ ID NO: 12, the VH CDR2 includes an amino acid sequence selected from the group consisting of SEQ ID NO: 16 and 49-54, the VH CDR3 includes the amino acid sequence selected from the group consisting of SEQ ID NO: 21, the VL CDR1 includes the amino acid sequence of SEQ ID NO: 28, the VL CDR2 includes the amino acid sequence of SEQ ID NO: 31, and the VL CDR3 includes an amino acid sequence selected from the group consisting of SEQ ID NO: 35, 63 and 64.

In some embodiments, the VH includes the amino acid sequence of SEQ ID NO: 3 or a sequence having at least 75%, 80%, 85%, 90%, 95% or 99% sequence identity to SEQ ID NO: 3, while retaining the VH CDRs of SEQ ID NO: 3 or PTM re-risked versions thereof. In some embodiments, the VL includes the amino acid sequence of SEQ ID NO: 4 or a sequence having at least 75%, 80%, 85%, 90%, 95% or 99% sequence identity to SEQ ID NO: 4, while retaining the VL CDRs of SEQ ID NO: 4 or PTM re-risked versions thereof. In some embodiments, the VH includes the amino acid sequence of SEQ ID NO: 3, and the VL includes the amino acid sequence of SEQ ID NO: 4.

Also provided, in some embodiments, are antibodies and antigen-binding fragments therefore that bind to the same epitope on FAPα as G52. Also provided, in some embodiments, are antibodies and antigen-binding fragments therefore that competes with G52 in binding to FAPα.

In some embodiments, provided is an antibody or antigen-binding fragment that is derived from antibody 130. In some embodiments, the VH CDR1 includes the amino acid sequence of SEQ ID NO: 67, the VH CDR2 includes an amino acid sequence selected from the group consisting of SEQ ID NO: 17 and 55, the VH CDR3 includes the amino acid sequence of SEQ ID NO: 22, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

In some embodiments, the VH CDR1 includes the amino acid sequence of SEQ ID NO: 12, the VH CDR2 includes the amino acid sequence of SEQ ID NO: 17, the VH CDR3 includes the amino acid sequence selected from the group consisting of SEQ ID NO: 22, the VL CDR 1 includes the amino acid sequence of SEQ ID NO: 27, the VL CDR2 includes the amino acid sequence of SEQ ID NO: 31, and the VL CDR3 includes the amino acid sequence of SEQ ID NO: 34.

In some embodiments, one or more of the CDRs is PTM (post-translational modification) de-risked. In some embodiments, the VH CDR1 includes the amino acid sequence of SEQ ID NO: 12, the VH CDR2 includes the amino acid sequence of SEQ ID NO: 17 or 55, the VH CDR3 includes the amino acid sequence selected from the group consisting of SEQ ID NO: 22, the VL CDR1 includes the amino acid sequence of SEQ ID NO: 27, the VL CDR2 includes the amino acid sequence of SEQ ID NO: 31, and the VL CDR3 includes an amino acid sequence selected from the group consisting of SEQ ID NO: 34, 61 and 62.

In some embodiments, the VH includes the amino acid sequence of SEQ ID NO: 5 or a sequence having at least 75%, 80%, 85%, 90%, 95% or 99% sequence identity to SEQ ID NO: 5, while retaining the VH CDRs of SEQ ID NO: 5 or PTM re-risked versions thereof. In some embodiments, the VL includes the amino acid sequence of SEQ ID NO: 6 or a sequence having at least 75%, 80%, 85%, 90%, 95% or 99% sequence identity to SEQ ID NO: 6, while retaining the VL CDRs of SEQ ID NO: 6 or PTM re-risked versions thereof. In some embodiments, the VH includes the amino acid sequence of SEQ ID NO: 5, and the VL includes the amino acid sequence of SEQ ID NO: 6.

Also provided, in some embodiments, are antibodies and antigen-binding fragments therefore that bind to the same epitope on FAPα as 130. Also provided, in some embodiments, are antibodies and antigen-binding fragments therefore that competes with 130 in binding to FAPα.

In some embodiments, provided is an antibody or antigen-binding fragment that is derived from antibody 137. In some embodiments, the VH CDR1 includes the amino acid sequence of SEQ ID NO: 67, the VH CDR2 includes an amino acid sequence selected from the group consisting of SEQ ID NO: 17 and 55, the VH CDR3 includes the amino acid sequence of SEQ ID NO: 23, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

In some embodiments, the VH CDR1 includes the amino acid sequence of SEQ ID NO: 12, the VH CDR2 includes the amino acid sequence of SEQ ID NO: 17, the VH CDR3 includes the amino acid sequence selected from the group consisting of SEQ ID NO: 23, the VL CDR 1 includes the amino acid sequence of SEQ ID NO: 27, the VL CDR2 includes the amino acid sequence of SEQ ID NO: 31, and the VL CDR3 includes the amino acid sequence of SEQ ID NO: 36.

In some embodiments, one or more of the CDRs is PTM (post-translational modification) de-risked. In some embodiments, the VH CDR1 includes the amino acid sequence of SEQ ID NO: 12, the VH CDR2 includes the amino acid sequence of SEQ ID NO: 17 or 55, the VH CDR3 includes the amino acid sequence selected from the group consisting of SEQ ID NO: 23, the VL CDR1 includes the amino acid sequence of SEQ ID NO: 27, the VL CDR2 includes the amino acid sequence of SEQ ID NO: 31, and the VL CDR3 includes an amino acid sequence selected from the group consisting of SEQ ID NO: 36, 65 and 66.

In some embodiments, the VH includes the amino acid sequence of SEQ ID NO: 7 or a sequence having at least 75%, 80%, 85%, 90%, 95% or 99% sequence identity to SEQ ID NO: 7, while retaining the VH CDRs of SEQ ID NO: 7 or PTM re-risked versions thereof. In some embodiments, the VL includes the amino acid sequence of SEQ ID NO: 8 or a sequence having at least 75%, 80%, 85%, 90%, 95% or 99% sequence identity to SEQ ID NO: 8, while retaining the VL CDRs of SEQ ID NO: 8 or PTM re-risked versions thereof. In some embodiments, the VH includes the amino acid sequence of SEQ ID NO: 7, and the VL includes the amino acid sequence of SEQ ID NO: 8.

Also provided, in some embodiments, are antibodies and antigen-binding fragments therefore that bind to the same epitope on FAPα as I37. Also provided, in some embodiments, are antibodies and antigen-binding fragments therefore that competes with I37 in binding to FAPα.

In some embodiments, provided is an antibody or antigen-binding fragment that is derived from antibody J40. In some embodiments, the VH CDR1 includes the amino acid sequence of SEQ ID NO: 67, the VH CDR2 includes an amino acid sequence selected from the group consisting of SEQ ID NO: 17 and 55, the VH CDR3 includes the amino acid sequence of SEQ ID NO: 25, the VL CDR1 includes the amino acid sequence of SEQ ID NO: 68, the VL CDR2 includes the amino acid sequence of SEQ ID NO: 31, and the VL CDR3 includes the amino acid sequence of SEQ ID NO: 69.

In some embodiments, the VH CDR1 includes the amino acid sequence of SEQ ID NO: 12, the VH CDR2 includes the amino acid sequence of SEQ ID NO: 17, the VH CDR3 includes the amino acid sequence selected from the group consisting of SEQ ID NO: 25, the VL CDR 1 includes the amino acid sequence of SEQ ID NO: 27, the VL CDR2 includes the amino acid sequence of SEQ ID NO: 31, and the VL CDR3 includes the amino acid sequence of SEQ ID NO: 34.

In some embodiments, one or more of the CDRs is PTM (post-translational modification) de-risked. In some embodiments, the VH CDR1 includes the amino acid sequence of SEQ ID NO: 12, the VH CDR2 includes the amino acid sequence of SEQ ID NO: 17 or 55, the VH CDR3 includes the amino acid sequence selected from the group consisting of SEQ ID NO: 25, the VL CDR1 includes the amino acid sequence of SEQ ID NO: 27, the VL CDR2 includes the amino acid sequence of SEQ ID NO: 31, and the VL CDR3 includes an amino acid sequence selected from the group consisting of SEQ ID NO: 34, 61 and 62.

In some embodiments, the VH includes the amino acid sequence of SEQ ID NO: 9 or a sequence having at least 75%, 80%, 85%, 90%, 95% or 99% sequence identity to SEQ ID NO: 9, while retaining the VH CDRs of SEQ ID NO: 9 or PTM re-risked versions thereof. In some embodiments, the VL includes the amino acid sequence of SEQ ID NO: 10 or a sequence having at least 75%, 80%, 85%, 90%, 95% or 99% sequence identity to SEQ ID NO: 10, while retaining the VL CDRs of SEQ ID NO: 10 or PTM re-risked versions thereof. In some embodiments, the VH includes the amino acid sequence of SEQ ID NO: 9, and the VL includes the amino acid sequence of SEQ ID NO: 10.

Also provided, in some embodiments, are antibodies and antigen-binding fragments therefore that bind to the same epitope on FAPα as J40. Also provided, in some embodiments, are antibodies and antigen-binding fragments therefore that competes with J40 in binding to FAPα.

In some embodiments, provided is an antibody or antigen-binding fragment that is derived from antibody 138. In some embodiments, the VH CDR1 includes the amino acid sequence of SEQ ID NO: 13, the VH CDR2 includes the amino acid sequence of SEQ ID NO: 18, the VH CDR3 includes the amino acid sequence selected from the group consisting of SEQ ID NO: 24, the VL CDR1 includes the amino acid sequence of SEQ ID NO: 29, the VL CDR2 includes the amino acid sequence of SEQ ID NO: 32, and the VL CDR3 includes the amino acid sequence of SEQ ID NO: 37.

In some embodiments, the VH includes the amino acid sequence of SEQ ID NO: 39 or a sequence having at least 75%, 80%, 85%, 90%, 95% or 99% sequence identity to SEQ ID NO: 39, while retaining the VH CDRs of SEQ ID NO: 39 or PTM re-risked versions thereof. In some embodiments, the VL includes the amino acid sequence of SEQ ID NO: 40 or a sequence having at least 75%, 80%, 85%, 90%, 95% or 99% sequence identity to SEQ ID NO: 40, while retaining the VL CDRs of SEQ ID NO: 40 or PTM re-risked versions thereof. In some embodiments, the VH includes the amino acid sequence of SEQ ID NO: 39, and the VL includes the amino acid sequence of SEQ ID NO: 40.

Also provided, in some embodiments, are antibodies and antigen-binding fragments therefore that bind to the same epitope on FAPα as I38. Also provided, in some embodiments, are antibodies and antigen-binding fragments therefore that competes with 138 in binding to FAPα.

In some embodiments, provided is an antibody or antigen-binding fragment that is derived from antibody J59. In some embodiments, the VH CDR1 includes the amino acid sequence of SEQ ID NO: 14, the VH CDR2 includes the amino acid sequence of SEQ ID NO: 19, the VH CDR3 includes the amino acid sequence selected from the group consisting of SEQ ID NO: 26, the VL CDR1 includes the amino acid sequence of SEQ ID NO: 30, the VL CDR2 includes the amino acid sequence of SEQ ID NO: 33, and the VL CDR3 includes the amino acid sequence of SEQ ID NO: 38.

In some embodiments, one or more of the CDRs is PTM (post-translational modification) de-risked. In some embodiments, the VH CDR1 includes the amino acid sequence of SEQ ID NO: 14, the VH CDR2 includes an amino acid sequence selected from the group consisting of SEQ ID NO: 19 and 56-58, the VH CDR3 includes the amino acid sequence selected from the group consisting of SEQ ID NO: 26, the VL CDR1 includes the amino acid sequence of SEQ ID NO: 30, the VL CDR2 includes the amino acid sequence of SEQ ID NO: 33, and the VL CDR3 includes the amino acid sequence of SEQ ID NO: 38.

In some embodiments, the VH includes the amino acid sequence of SEQ ID NO: 41 or a sequence having at least 75%, 80%, 85%, 90%, 95% or 99% sequence identity to SEQ ID NO: 41, while retaining the VH CDRs of SEQ ID NO: 41 or PTM re-risked versions thereof. In some embodiments, the VL includes the amino acid sequence of SEQ ID NO: 42 or a sequence having at least 75%, 80%, 85%, 90%, 95% or 99% sequence identity to SEQ ID NO: 42, while retaining the VL CDRs of SEQ ID NO: 42 or PTM re-risked versions thereof. In some embodiments, the VH includes the amino acid sequence of SEQ ID NO: 41, and the VL includes the amino acid sequence of SEQ ID NO: 42.

Also provided, in some embodiments, are antibodies and antigen-binding fragments therefore that bind to the same epitope on FAPα as J59. Also provided, in some embodiments, are antibodies and antigen-binding fragments therefore that competes with J59 in binding to FAPα.

Also provided, in some embodiments, are antibodies and antigen-binding fragments that include CDR sequences derived from the presently disclosed CDR sequences, with one, two or three amino acid substitutions, deletions, and/or additions.

### Multi-Functional Molecules

Multi-functional molecules that include an antibody or antigen-binding fragment specific to FAPα, such as those disclosed herein, and one or more antibody or antigen-binding fragment having specificity to a second antigen.

In some embodiments, the second antigen is a protein expressed on an immune cell, such as a T cell, a B cell, a monocyte, a macrophage, a neutrophil, a dendritic cell, a phagocyte, a natural killer cell, an eosinophil, a basophil, and a mast cell.

In some embodiments, the second antigen is CD3, CD47, PD1, PD-L1, LAG3, TIM3, CTLA4, VISTA, CSFR1, A2AR, CD73, CD39, CD40, CEA, HER2, CMET, 4-1BB, OX40, SIRPA CD16, CD28, ICOS, CTLA4, BTLA, TIGIT, HVEM, CD27, VEGFR, or VEGF.

Different formats of bispecific antibodies are also provided. In some embodiments, each of the anti-FAPα fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to FAPα, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bi-functional molecules can combine the immune checkpoint blocking effect with tumor site local immune modulation.

### Chimeric Antigen Receptors

Also provided, in one embodiment, is a chimeric antigen receptor (CAR) that includes the antibody or fragment thereof of the present disclosure as a targeting unit. In some embodiments, the CAR includes an antibody or fragment thereof of the present disclosure, a transmembrane domain, a costimulatory domain, and a CD3ξ intracellular domain.

A transmembrane domain can be designed to be fused to the extracellular domain which includes the antibody or fragment, optionally through a hinge domain. It can similarly be fused to an intracellular domain, such as a costimulatory domain. In some embodiments, the transmembrane domain can include the natural transmembrane region of a costimulatory domain (e.g., the TM region of a CD28T or 4-1BB employed as a costimulatory domain) or the natural transmembrane domain of a hinge region (e.g., the TM region of a CD8 alpha or CD28T employed as a hinge domain).

In some embodiments, the transmembrane domain can include a sequence that spans a cell membrane, but extends into the cytoplasm of a cell and/or into the extracellular space. For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain”.

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the CAR further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 1 la/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the cytoplasmic portion of the CAR also includes a signaling/activation domain. In one embodiment, the signaling/activation domain is the CD38 domain, or is an amino acid sequence having at least about 80%, 85%, 90%, 95%, 98% or 99% sequence identity to the CD38 domain.

Polynucleotides, mRNA, and Methods of Expressing or Preparing Antibodies

The present disclosure also provides polynucleotides or nucleic acid molecules encoding the antibodies, variants or derivatives thereof of the disclosure, or the CAR. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, the polynucleotide is an mRNA molecule. In some embodiments, the mRNA can be introduced into a target cell for expressing the antibody or fragment thereof.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of antibody-coding mRNA, a DNA template is transcribed in vitro. A suitable DNA template typically has a promoter, for example a T3, T7 or SP6 promoter, for in vitro transcription, followed by desired nucleotide sequence for desired antibody encoding (e.g., heavy chain or light chain encoding) mRNA and a termination signal.

Desired antibody encoding (e.g., heavy chain or light chain encoding) mRNA sequence may be determined and incorporated into a DNA template using standard methods. For example, starting from a desired amino acid sequence (e.g., a desired heavy chain or light chain sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA may be synthesized as unmodified or modified mRNA. Typically, mRNAs are modified to enhance stability. Modifications of mRNA can include, for example, modifications of the nucleotides of the RNA. A modified mRNA can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, the mRNAs (e.g., heavy chain and light chain encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, the mRNAs (e.g., heavy chain and light chain encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs (e.g., heavy chain and light chain encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs (e.g., heavy chain and light chain encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5)A and G(5)ppp(5′)G.

In some embodiments, the mRNAs (e.g., heavy chain and light chain encoding mRNAs) include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10 to 125 adenosine nucleotides, 10 to 100 adenosine nucleotides, about 10 to 75 adenosine nucleotides, about 20 to 70 adenosine nucleotides, or about 20 to 60 adenosine nucleotides). In some embodiments, antibody encoding mRNAs (e.g., heavy chain and light chain encoding mRNAs) include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs (e.g., heavy chain and light chain encoding mRNAs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA (e.g., heavy chain and light chain encoding mRNAs) includes a sequence encoding a signal peptide, such as those described herein. In particular embodiments, a signal peptide derived from human growth hormone (hGH) is incorporated in the 5′ region. Typically, a signal peptide encoding sequence is linked, directly or indirectly, to the heavy chain or light chain encoding sequence at the N-terminus.

The present technology may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bi-specific antibodies.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment and Uses

As described herein, the antibodies, variants, derivatives or antibody-drug conjugates of the present disclosure may be used in certain treatment and diagnostic methods.

The present disclosure is further directed to antibody-based therapies which involve administering the antibodies, fragments, or antibody-drug conjugates of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. As provided above, FAPα can be overexpressed in tumor cells, in particular liver, gastric, pancreatic, esophageal, ovarian, and lung tumors. Inhibition of FAPα has been shown to be useful for treating the tumors.

Accordingly, in some embodiments, provided are methods for treating a cancer in a patient in need thereof. The method, in one embodiment, entails administering to the patient an effective amount of an antibody, fragment, or antibody-drug conjugate of the present disclosure. In some embodiments, at least one of the cancer cells (e.g., stromal cells) in the patient over-express FAPα.

Cellular therapies, such as chimeric antigen receptor (CAR) T-cell therapies, are also provided in the present disclosure. A suitable cell can be used, that is transduced with a vector that encodes, or put in contact with, an CAR that includes an anti-FAPα antibody of the present disclosure (or alternatively engineered to express an anti-FAPα antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

FAPα is involved in the control of fibroblast growth or epithelial-mesenchymal interactions during development, tissue repair, and epithelial carcinogenesis. It was observed that high expression of FAPα in RA (rheumatoid arthritis) fibroblast-like synoviocytes (FLSs) is associated with an invasive phenotype of FLSs accompanied by high proliferation and destruction of the extracellular matrix. Therefore, FAPα can be considered as a therapeutic target to inhibit the destructive potential of FLSs.

Also provided are methods and uses for treating inflammatory conditions or autoimmune disease. In some embodiments, the inflammatory disease or condition to be treated by the disclosed antibodies, fragments and compositions includes one or more of Alzheimer's disease, Addison's disease, atherosclerosis, ankylosing spondylitis, arthritis, osteoarthritis (OA), rheumatoid arthritis (RA), psoriatic arthritis (PA), ankylosing spondylitis, asthma, atherosclerosis, chronic obstructive pulmonary disease (COPD), Crohn's disease, colitis, dermatitis, diverticulitis, fibromyalgia, hepatitis, irritable bowel syndrome (IBS), systemic lupus erythematous (SLE), nephritis, Parkinson's disease (PD), vasculitis, and ulcerative colitis.

In some embodiments, the autoimmune disease or condition to be treated by the disclosed antibodies, fragments and compositions includes one or more of alopecia areata, autoimmune hemolytic anemia, autoimmune hepatitis, dermatomyositis, diabetes (type 1), celiac disease, autoimmune juvenile idiopathic arthritis, glomerulonephritis, Graves' disease, Guillain-Barré syndrome, idiopathic thrombocytopenia purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, Hashimoto's thyroiditis, autoimmune uveitis, vitiligo, and granulomatosis with polyangiitis (Wegener's).

Rheumatoid arthritis (RA) is a long-term autoimmune disorder that primarily affects joints. It typically results in warm, swollen, and painful joints. Pain and stiffness often worsen following rest. Most commonly, the wrist and hands are involved, with the same joints typically involved on both sides of the body. The disease may also affect other parts of the body. While the cause of rheumatoid arthritis is not clear, it is believed to involve a combination of genetic and environmental factors. The underlying mechanism involves the body's immune system attacking the joints. This results in inflammation and thickening of the joint capsule. The goals of treatment are to reduce pain, decrease inflammation, and improve a person's overall functioning. Pain medications, steroids, and NSAIDs are frequently used to help with symptoms. A group of medications called disease-modifying antirheumatic drugs (DMARDs), such as hydroxychloroquine and methotrexate, may be used to try to slow the progression of disease.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibody, fragment, or antibody-drug conjugate or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracisternally, intravaginally, intraperitoneally, topically (as by powders, ointments, drops or transdermal patch), buccally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies, fragments, or antibody-drug conjugates of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies, fragments, or antibody-drug conjugates of the present disclosure is typically 0.001 mg/kg to 100 mg/kg of the patient's body weight, between 0.01 mg/kg and 20 mg/kg of the patient's body weight, or 0.5 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, fragment, or antibody-drug conjugate, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Full Human Naïve Phage Library Panning and Screening

This example screened for full human anti-FAPα antibodies from a human Naïve phage library.

Antigen: human FAPα-His tag (Sino biological).

Preparation of full human naïve phage library: The phage library was constructed by using phagemid vectors which consisted of antibody gene fragments that were amplified from PBMCs of healthy human subjects. The library format is Fab phage library. The library size was 5.2×10¹⁰.

Phage library solid phase panning and solution panning against human FAPα protein. For solution panning, the phage libraries were firstly performed negative screening by incubating with BSA-coated streptavidin Dynabeads. The resulting phages were incubated with Biotinylated-FAPα-His tag protein and washed by Kingfihser magnetic beads system. The binders were eluted by trypsin. For solid phase panning, the phage libraries were firstly performed negative blocking by 5% PBSM. The resulting phages were incubated with FAPα-His tag protein and washed by PBST+PBS. The binders were eluted by trypsin.

The solid phase panning was combined with solution panning to comprehensively screening the potential binders. The eluted phages were subsequently tested for their titer to bind antigen and co-cultured with E. coli. There were five rounds of panning and screening. The titers of output 4 and output 5 were significantly increased.

Single clones were cherrypicked from output 4 and 5 and then cultured in 96 deep well plate. The culture supernatant was subject to IgG concentration and antigen binding titer evaluation. A total of 390 positive clones were selected and subject to sequencing. Post sequence analysis identified 176 unique sequences. All these clones were subjected to ELISA binding analysis. Seven top sequences were identified see below table. Their sequences are provided in Table 1A below.

TABLE 1A
Candidate antibody sequences

| | | SEQ |
|---|---|---|
| | Sequence (CDRs according to | ID |
| Clone | Kabat system are underlined) | NO: |
| G14 VH | QVQLLESGGGVVQPGRSLRLSCAASGFTFS | 1 |
| | SYAMHWVRQAPGKGLEWVAFISHDGNRNSY | |
| | ADSVRGRFTISRDNSKNTLYLQMNSLRAED | |
| | TAVYYCARDLGYYNGFSYWGQGTLVTVSA | |
| G14 VL | DIVMTQSPSSVSASVGDRVTITCRASQGIS | 2 |
| | SWLAWYQQKPGKAPKLLIYAASSLQSGVPS | |
| | RFSGSGSGTDFTLTISSLQPEDFATYYCQQ | |
| | ANSFPPTFGPGTKVDIK | |
| G52 VH | EVQLLESGGDLVQPGGSLRLSCAASGFTFS | 3 |
| | SYAMSWVRQAPGKGLEWVSGSNGGDSIAYA | |
| | DISVKGRFTVFRDNAKNTLYLQMNSLRAED | |
| | TAIYYCARGNYYAHDDWGQGTLVTVSA | |
| G52 VL | DIQLTQSPSSVSASVGDRVTITCRASQGVG | 4 |
| | SWLAWYQQKPGKPPKLLIYAASSLQSGVPS | |
| | RFSGSGSGTDFTLTISSLQPEDFATYYCQQ | |
| | ANSFPLTFGGGTKVEIK | |
| I30 VH | EVQLLESGGGLVQPGGSLRLSCAASGFTFS | 5 |
| | SYAMSWVRQAPGKGLEWVSAISGSGGSTYY | |
| | ADSVKGRFTISRDNSKNTLYLQMNSLRAED | |
| | TAVYYCAKDLSGGTSFDYWGQGTLVTVSA | |
| I30 VL | AIRLTQSPSSVSASVGDRVTITCRASQGIS | 6 |
| | SWLAWYQQKPGKAPKLLIYAASSLQSGVPS | |
| | RFSGSGSGTDFTLTISSLQPEDFATYYCQQ | |
| | ANSFPPTFGGGTKVEIK | |
| I37 VH | QLQLVESGGGLVQPGGSLRLSCAASGFTFS | 7 |
| | SYAMSWVRQAPGKGLEWVSAISGSGGSTYY | |
| | ADSVKGRFTISRDNSKNTLYLQMNSLRAED | |
| | TAVYYCARDKPWGGSFDYWGQGTLVTVSA | |
| I37 VL | DIQLTQSPSSLSASVGDRVTITCRASQGIS | 8 |
| | SWLAWYQQKPGKAPKLLIYAASSLQSGVPS | |
| | RFSGSGSGTDFTLTISSLQPEDFATYYCQQ | |
| | ANSFPVTFGPGTKVDIK | |
| I38 VH | EVQLVQSGAEVKEPGVSVKVSCKASGYTFA | 39 |
| | SYGLSWVRLAPGQGLQWMGWISPYTPNPIY | |
| | APNFRDKVILTTDRSTNTAYLEVRSLRSDD | |
| | TGLYYCARNHWVGRPTRAFDLWGQGTMVTV | |
| | SA | |
| I38 VL | EIVLTQSPGTLSLSPGERATLSCRASQTVS | 40 |
| | ITHLAWYQQKPGQAPRLLTYGASARATGIP | |
| | DRFSVSGSGTDFTLTITRLEPEDFAVYYCQ | |
| | HYGSSITFGGGTKVEIK | |
| J40 VH | EVQLLESGGGLVQPGGSLRLSCAASGFTFS | 9 |
| | SYAMSWVRQAPGKGLEWVSAISGSGGSTYY | |
| | ADSVKGRFTISRDNSKNTLYLQMNSLRTDD | |
| | TAVYYCARDLTTSTGWESWGQGTLVTVSA | |
| J40 VL | NIQMTQSPSSVSASVGDRVTITCRASQGIS | 10 |
| | SWLAWYQQKPGKAPKLLIYAASSLQSGVPS | |
| | RFSGSGSGTDFTLTISSLQPEDFATYYCQQ | |
| | ANSFPPTFGQGTKLEIK | |
| J59 VH | QVQLLESGGGVVQPGRSLRLSCAASGFTFS | 41 |
| | SYGMHWVRQAPGKGLEWVAVISYDGSNKYY | |
| | ADSVKGRFTISRDNSKNTLYLQMNSLRADD | |
| | TAVYYCARESYTGLGSFLHWGQGTLVTVSA | |
| J59 VL | NIQLTQSPSSLSASVGDRVTITCRASQTIS | 42 |
| | NYLNWYQQKPGKAPKLLISAASSLQGGVPS | |
| | RFSGSGSGTDFTLAISSLQPEDSATYYCQY | |
| | WGTFGQGTKLEIK | |

The CDR sequences of these antibodies (according to Kabat system) are listed in Table 1B below. Also shown are their variants in which potential sites for post-translational modification (PTM) are mutated to biological equivalents (e.g., NG=>NA, NG=>QG, NS=>NA, NS=>QS, DG=>DA and DS=>DA). These substitutions are contemplated to retain the biological activities of the antibodies while preventing PTM, to facilitate manufacturing.

TABLE 1B
CDR Sequences

| | | SEQ | | SEQ | | SEQ |
|---|---|---|---|---|---|---|
| | VH | ID | VH | ID | VH | ID |
| Ab | CDR1 | NO: | CDR2 | NO: | CDR3 | NO: |
| G14 | SYAMH | 11 | FISHDGNRN | 15 | DLGYYNGFSY | 20 |
| | | | SYADSVRG | | | |
| | | | FISHDANRN | 43 | DLGYYNAFSY | 59 |
| | | | SYADSVRG | | | |
| | | | FISHDGNRN | 44 | DLGYYQGFSY | 60 |
| | | | AYADSVRG | | | |
| | | | FISHDGNRQ | 45 | | |
| | | | SYADSVRG | | | |
| | | | FISHDGNRN | 46 | | |
| | | | SYADAVRG | | | |
| | | | FISHDANRN | 47 | | |
| | | | AYADAVRG | | | |
| | | | FISHDANRQ | 48 | | |
| | | | SYADAVRG | | | |
| G52 | SYAMS | 12 | GISNGGDSI | 16 | GNYYAHDD | 21 |
| | | | AYADSVKG | | | |
| | | | GISNAGDSI | 49 | | |
| | | | AYADSVKG | | | |
| | | | GISQGGDSI | 50 | | |
| | | | AYADSVKG | | | |
| | | | GISNGGDAI | 51 | | |
| | | | AYADSVKG | | | |
| | | | GISNGGDSI | 52 | | |
| | | | AYADAVKG | | | |
| | | | GISNAGDAI | 53 | | |
| | | | AYADAVKG | | | |
| | | | GISQGGDAI | 54 | | |
| | | | AYADAVKG | | | |
| I30 | SYAMS | 12 | AISGSGGST | 17 | DLSGGTSFDY | 22 |
| | | | YYADSVKG | | | |
| | | | AISGSGGST | 55 | | |
| | | | YYADAVKG | | | |
| I37 | SYAMS | 12 | AISGSGGST | 17 | DKPWGGSFDY | 23 |
| | | | YYADSVKG | | | |
| | | | AISGSGGST | 55 | | |
| | | | YYADAVKG | | | |
| I38 | SYGLS | 13 | WISPYTPNP | 18 | NHWVGRPTRAFDL | 24 |
| | | | IYAPNFRD | | | |
| J40 | SYAMS | 12 | AISGSGGST | 17 | DLTTSTGWES | 25 |
| | | | YYADSVKG | | | |
| | | | AISGSGGST | 55 | | |
| | | | YYADAVKG | | | |
| J59 | SYGMH | 14 | VISYDGSNK | 19 | ESYTGLGSFLH | 26 |
| | | | YYADSVKG | | | |
| | | | VISYDASNK | 56 | | |
| | | | YYADSVKG | | | |
| | | | VISYDGSNK | 57 | | |
| | | | YYADAVKG | | | |
| | | | VISYDASNK | 58 | | |
| | | | YYADAVKG | | | |
| | | SEQ | | SEQ | | SEQ |
| | VL | ID | VL | ID | VL | ID |
| Ab | CDR1 | NO: | CDR2 | NO: | CDR3 | NO: |
| G14 | RASQG | 27 | AASSLQS | 31 | QQANSFPPT | 34 |
| | ISSWL | | | | QQANAFPPT | 61 |
| | A | | | | QQAQSFPPT | 62 |
| G52 | RASQG | 28 | AASSLQS | 31 | QQANSFPLT | 35 |
| | VGSWL | | | | QQANAFPLT | 63 |
| | A | | | | QQAQSFPLT | 64 |
| I30 | RASQG | 27 | AASSLQS | 31 | QQANSFPPT | 34 |
| | ISSWL | | | | QQANAFPPT | 61 |
| | A | | | | QQAQSFPPT | 62 |
| I37 | RASQG | 27 | AASSLQS | 31 | QQANSFPVT | 36 |
| | ISSWL | | | | QQANAFPVT | 65 |
| | A | | | | QQAQSFPVT | 66 |
| I38 | RASQT | 29 | GASARAT | 32 | QHYGSSIT | 37 |
| | VSITH | | | | | |
| | LA | | | | | |
| J40 | RASQG | 27 | AASSLQS | 31 | QQANSFPPT | 34 |
| | ISSWL | | | | QQANAFPPT | 61 |
| | A | | | | QQAQSFPPT | 62 |
| J59 | RASQT | 30 | AASSLQG | 33 | QYWGT | 38 |
| | ISNYL | | | | | |
| | N | | | | | |

Underlined residues are substituted ones to prevent post-translational modifications (PTM)

Some of these antibodies, in particular G14, G52, 130, 137 and J40, have highly homologous VH CDR1 and VL CDR sequences. It is contemplated that these CDR sequences are interchangeable. Their alignment and consensus sequences are shown in Table 1C.

TABLE 1C
Consensus CDRs

| | | | SEQ |
|---|---|---|---|
| | Consensus | | ID |
| CDR | (examples) | Definitions | NO: |
| VH | SYAMX | X is H or S | 67 |
| CDR1 | (SYAMH | | 11 |
| | SYAMS) | | 12 |
| VH | (FISHDGNRNSYADSVRG | | 15 |
| CDR2 | FISHDANRNSYADSVRG | | 43 |
| | FISHDGNRNAYADSVRG | | 44 |
| | FISHDGNRQSYADSVRG | | 45 |
| | FISHDGNRNSYADAVRG | | 46 |
| | FISHDANRNAYADAVRG | | 47 |
| | FISHDANRQSYADAVRG | | 48 |
| | GISNGGDSIAYADSVKG | | 16 |
| | GISNAGDSIAYADSVKG | | 49 |
| | GISQGGDSIAYADSVKG | | 50 |
| | GISNGGDAIAYADSVKG | | 51 |
| | GISNGGDSIAYADAVKG | | 52 |
| | GISNAGDAIAYADAVKG | | 53 |
| | GISQGGDAIAYADAVKG | | 54 |
| | AISGSGGSTYYADSVKG | | 17 |
| | AISGSGGSTYYADAVKG) | | 55 |
| VH | (DLGYYNGFSY | | 20 |
| CDR3 | DLGYYNAFSY | | 59 |
| | DLGYYQGFSY | | 60 |
| | GNYYAHDD | | 21 |
| | DLSGGTSFDY | | 22 |
| | DKPWGGSFDY | | 23 |
| | DLTTSTGWES) | | 25 |
| VL | RASQGX₁X₂SWLA | X₁ is I or V, | 68 |
| CDR1 | (RASQGISSWLA | X₂ is G or S | 27 |
| | RASQGVGSWLA) | | 28 |
| VL | AASSLQS | | 31 |
| CDR2 | | | |
| VL | QQAX₁X₂FPX₃T | X₁ is N or W, | 69 |
| CDR3 | (QQANSFPPT | X₂ is A or S, | 34 |
| | QQANAFPPT | X₃ is L, P or V | 61 |
| | QQAQSFPPT | | 62 |
| | QQANSFPLT | | 35 |
| | QQANAFPLT | | 63 |
| | QQAQSFPLT | | 64 |
| | QQANSFPVT | | 36 |
| | QQANAFPVT | | 65 |
| | QQAQSFPVT) | | 66 |

### Example 2: Binding Activity to Human and Mouse FAPα Antigen

This example tested the binding activities of the antibodies to human and mouse FAPα protein.

### 2.1 ELISA Binding to Human FAPα.

To evaluate the binding activity of clones, the tested antibodies were subjected to ELISA test.

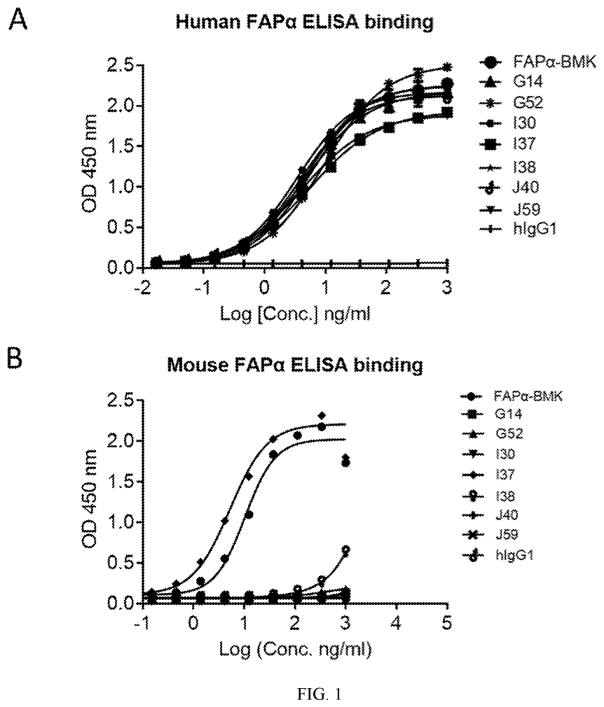

Briefly, microtiter plates were coated with human FAPα-His protein at 0.5 ug/ml in PBS, 100 μl/well at 4° C. overnight, then blocked with 150 μl/well of 1% BSA. Three-fold dilutions of tested antibodies starting from 1 μg/ml were added to each well and incubated for 1 hour at 37° C. The plates were washed with PBS/Tween and then incubated with Anti-Human IgG (H&L) (GOAT) Antibody Peroxidase Conjugated for 30 mins at 37° C. After washing, the plates were developed with TMB substrate and analyzed by spectrophotometer at OD 450 nm. As shown in FIG. 1A and Table 2, all of G14, G52, 130, 137, 138, J40, and J59 clones bound to human FAPα with high affinity. FAP-4B9 clone from Roche was served as positive control which labeled as FAPα-BMK.

### 2.1 ELISA Binding to Mouse FAPα

To evaluate the binding activity of clones, the tested antibodies were subjected to ELISA test.

Briefly, microtiter plates were coated with mouse FAPα-His protein at 0.5 ug/ml in PBS, 100 μl/well at 4° C. overnight, then blocked with 150 μl/well of 1% BSA. Three-fold dilutions of tested antibodies starting from 1 μg/ml were added to each well and incubated for 1 hour at 37° C. The plates were washed with PBS/Tween and then incubated with Anti-Human IgG (H&L) (GOAT) Antibody Peroxidase Conjugated for 30 mins at 37° C. After washing, the plates were developed with TMB substrate and analyzed by spectrophotometer at OD 450 nm. As shown in FIG. 1B and Table 2, only 137 and FAPα-BMK bound to mouse FAPα with high affinity.

TABLE 2
Binding activity to human and mouse FAPα

| | Human FAPα | Mouse FAPα |
|---|---|---|
| Abs | EC50 (ng/ml) | EC50 (ng/ml) |
| FAPα-BMK | 5.553 | 10.3 |
| G14 | 4.643 | ND |
| G52 | 9.483 | ND |
| I30 | 3.416 | ND |
| I37 | 5.396 | 5.202 |
| I38 | 4.894 | ND |
| J40 | 3.869 | ND |
| J59 | 3.943 | ND |

### 2.2 Cell-Based Binding to Human FAPα

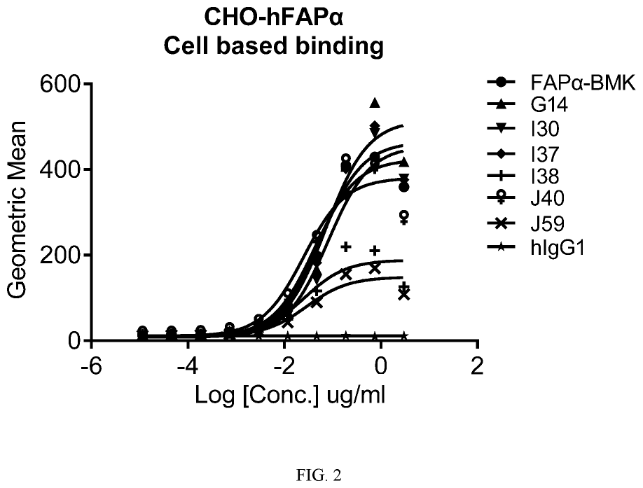

To evaluate the cell-based binding property to human FAPα, the tested antibodies were analyzed for their binding to CHO-K1-hFAPα by FACS. A total number of 1×10⁵ CHO-K1-hFAPα cells in each well were incubated with 4-fold serial diluted antibodies starting from 3 μg/ml for 30 minutes at 4° C. in FACS buffer. After wash by FACS buffer, PE conjugated-anti-human IgG antibody was added to each well and incubated at 4° C. for 30 minutes. After wash, MFI of PE was evaluated by MACSQuant Analyzer 16. As shown in FIG. 2 and Table 3, the G14, 130, 137, and J40 antibodies showed comparable binding abilities to “FAPα BMK antibody,” a positive reference, while the 138 and J59 clones lost binding activity dramatically.

TABLE 3
The binding activity to CHO-K1-hFAPα cells

| Abs | EC50 (ng/ml) | Cmax |
|---|---|---|
| FAPα-BMK | 0.043 | 423.6 |
| G14 | 0.073 | 515.1 |
| I30 | 0.080 | 455.2 |
| I37 | 0.057 | 464.7 |
| J40 | 0.025 | 380.6 |
| I38 | 0.026 | 188.6 |
| J59 | 0.029 | 148.7 |

### 2.3 Protein Full Kinetic for Human FAPα

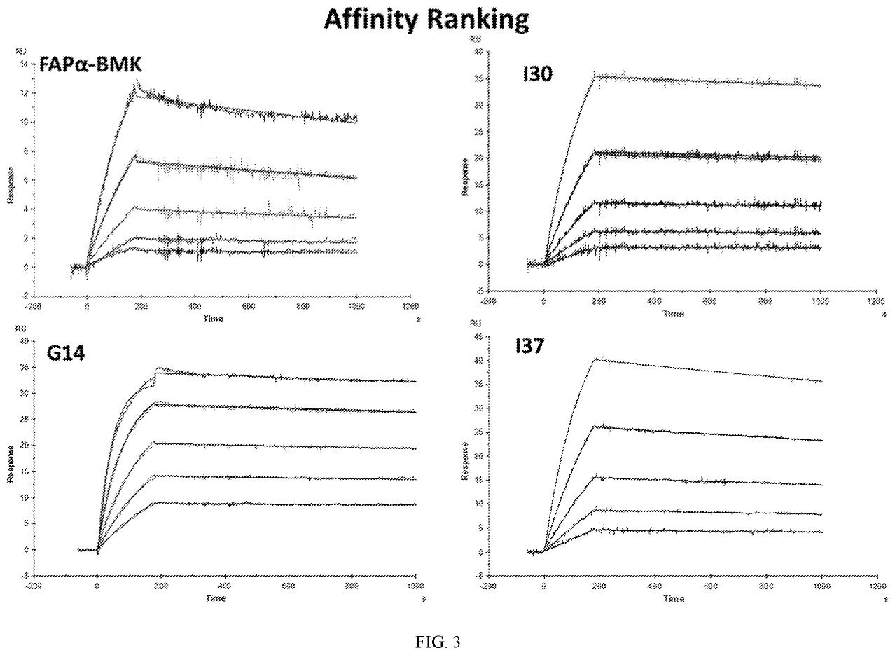

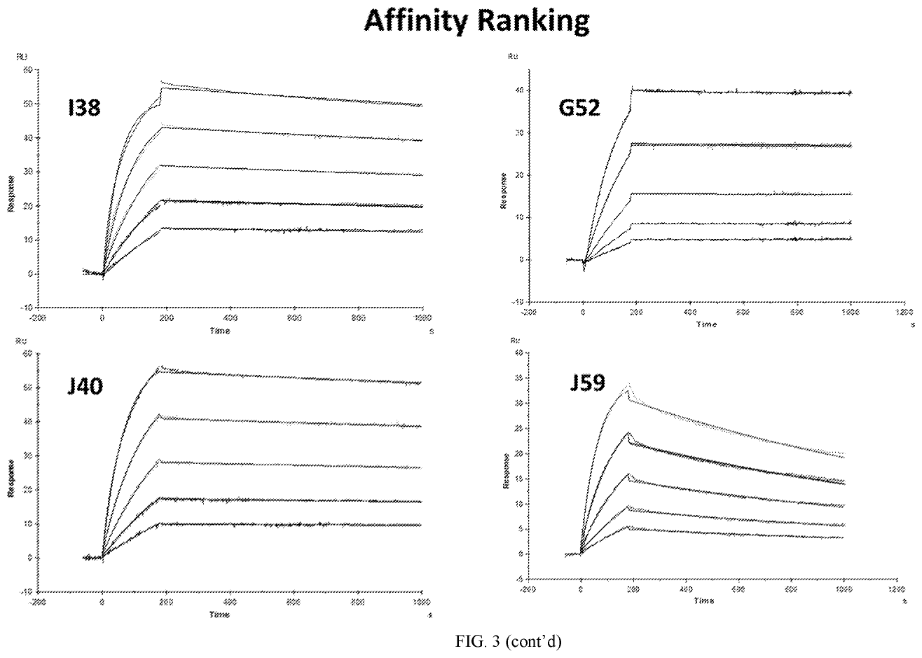

The binding of the tested antibodies to recombinant FAPα protein (human FAPα-his tag) was tested with Biacore using a capture method. The mAbs were captured using Protein A chip. A serial dilution of human FAPα-his tag protein was injected over captured antibody for 3 mins at a flow rate of 30 μl/min. The antigen was allowed to dissociate for 800s. All the experiments were carried out on a Biacore T200. Data analysis was carried out using the Biacore T200 evaluation software. The results are shown in FIG. 3 and Table 4 below.

TABLE 4
Full kinetic measured by Biacore

| | Human FAPα-His | | |
|---|---|---|---|
| Abs | ka (1/Ms) | kd (1/s) | KD (M) |
| FAPα-BMK | 3.002 × 10⁴ | 2.082 × 10⁻⁴ | 6.937 × 10⁻⁹ |
| G14 | 1.196 × 10⁵ | 6.237 × 10⁻⁵ | 5.216 × 10⁻¹⁰ |
| I30 | 1.367 × 10⁵ | 6.200 × 10⁻⁵ | 4.536 × 10⁻¹⁰ |
| I37 | 1.799 × 10⁵ | 1.537 × 10⁻⁴ | 8.547 × 10⁻¹⁰ |
| I38 | 1.030 × 10⁵ | 1.156 × 10⁻⁴ | 1.122 × 10⁻⁹ |
| J40 | 7.216 × 10⁴ | 7.309 × 10⁻⁵ | 1.013 × 10⁻⁹ |
| J59 | 3.333 × 10⁵ | 5.939 × 10⁻⁴ | 1.782 × 10⁻⁹ |
| G52 | 4.051 × 10⁴ | 1.805 × 10⁻⁵ | 4.456 × 10⁻¹⁰ |

### Example 3: Epitope Binning by Competitive ELISA

Competitive ELISA was carried out to classify the FAPα mAbs based on the binding epitope to human FAPα.

Briefly, microtiter plates were coated with human FAPα proteins at 0.5 μg/ml in PBS, 100 μl/well at 4° C. overnight, then blocked with 150 μl/well of 1% BSA. Serial dilutions of chimeric antibodies as well as biotin-conjugated reference mAb were added to each well and incubated for 1 hour at RT. The plates were washed with PBS/Tween and then incubate with streptavidin-HRP for 15 mins at RT. After washing, the plates were developed with TMB substrate and analyzed by spectrophotometer at OD 450 nm. According to the competition ability to reference mAbs (Benchmark, or “BMK”), all tested FAPα mAbs binds to the similar epitope as shown in Table 5.

TABLE 5
Epitope binning test by ELISA.

| Competing | FAP- | FAP-I30- | FAP-I37- | FAP-I38- | FAPα | FAP- | FAP-J40- |
|---|---|---|---|---|---|---|---|
| Ab | G14-Bio | Bio | Bio | Bio | (BMK)-Bio | G52-Bio | Bio |
| 0 | 2.149 | 1.490 | 1.721 | 1.212 | 1.256 | 1.264 | 1.203 |
| FAP-G14 | 0.209 | 0.241 | 0.457 | 0.658 | 0.399 | 0.195 | 0.293 |
| FAP-I30 | 0.129 | 0.143 | 0.219 | 0.324 | 0.151 | 0.146 | 0.160 |
| FAP-I37 | 0.224 | 0.239 | 0.602 | 0.703 | 0.460 | 0.180 | 0.314 |
| FAP-I38 | 0.149 | 0.220 | 0.282 | 0.396 | 0.154 | 0.176 | 0.226 |
| FAPα (BMK) | 0.135 | 0.155 | 0.230 | 0.265 | 0.171 | 0.169 | 0.179 |
| FAP-G52 | 0.358 | 0.302 | 0.399 | 0.466 | 0.304 | 0.283 | 0.298 |
| FAP-J40 | 0.167 | 0.218 | 0.435 | 0.527 | 0.343 | 0.206 | 0.261 |

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof which has specificity to the human fibroblast activation protein alpha (FAPα) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein:
the VH CDR1 comprises the amino acid sequence of SEQ ID NO: 67;
the VH CDR2 comprises an amino acid sequence selected from the group consisting of SEQ ID NO: 15-17 and 43-55;
the VH CDR3 comprises an amino acid sequence selected from the group consisting of SEQ ID NO: 20-23, 25, 59 and 60;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

2. The antibody or antigen-binding fragment thereof of claim 1, wherein:
the VH CDR1 comprises the amino acid sequence of SEQ ID NO: 67;
the VH CDR2 comprises an amino acid sequence selected from the group consisting of SEQ ID NO: 15 and 43-48;
the VH CDR3 comprises an amino acid sequence selected from the group consisting of SEQ ID NO: 20, 59 and 60;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

3. The antibody or antigen-binding fragment thereof of claim 1, wherein:
the VH CDR1 comprises the amino acid sequence of SEQ ID NO: 11;
the VH CDR2 comprises an amino acid sequence selected from the group consisting of SEQ ID NO: 15 and 43-48;
the VH CDR3 comprises an amino acid sequence selected from the group consisting of SEQ ID NO: 20, 59 and 60;
the VL CDR1 comprises the amino acid sequence of SEQ ID NO: 27;
the VL CDR2 comprises the amino acid sequence of SEQ ID NO: 31; and
the VL CDR3 comprises an amino acid sequence selected from the group consisting of SEQ ID NO: 34, 61 and 62.

4. The antibody or antigen-binding fragment thereof of claim 3, wherein the VH comprises the amino acid sequence of SEQ ID NO: 1 and the VL comprises the amino acid sequence of SEQ ID NO: 2.

5. The antibody or antigen-binding fragment thereof of claim 1, wherein:
the VH CDR 1 comprises the amino acid sequence of SEQ ID NO: 67;
the VH CDR2 comprises an amino acid sequence selected from the group consisting of SEQ ID NO: 16 and 49-54;
the VH CDR3 comprises the amino acid sequence of SEQ ID NO: 21;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

6. The antibody or antigen-binding fragment thereof of claim 5, wherein:
the VH CDR1 comprises the amino acid sequence of SEQ ID NO: 12;
the VH CDR2 comprises an amino acid sequence selected from the group consisting of SEQ ID NO: 16 and 49-54;
the VH CDR3 comprises the amino acid sequence of SEQ ID NO: 21;
the VL CDR1 comprises the amino acid sequence of SEQ ID NO: 28;
the VL CDR2 comprises the amino acid sequence of SEQ ID NO: 31; and
the VL CDR3 comprises an amino acid sequence selected from the group consisting of SEQ ID NO: 35, 63 and 64.

7. The antibody or antigen-binding fragment thereof of claim 6, wherein the VH comprises the amino acid sequence of SEQ ID NO: 3 and the VL comprises the amino acid sequence of SEQ ID NO: 4.

8. The antibody or antigen-binding fragment thereof of claim 1, wherein:
the VH CDR1 comprises the amino acid sequence of SEQ ID NO: 67;
the VH CDR2 comprises the amino acid sequence of SEQ ID NO: 17 or 55;
the VH CDR3 comprises the amino acid sequence of SEQ ID NO: 22;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

9. The antibody or antigen-binding fragment thereof of claim 8, wherein:
the VH CDR1 comprises the amino acid sequence of SEQ ID NO: 12;
the VH CDR2 comprises the amino acid sequence of SEQ ID NO: 17 or 55;
the VH CDR3 comprises the amino acid sequence of SEQ ID NO: 22;
the VL CDR1 comprises the amino acid sequence of SEQ ID NO: 27;
the VL CDR2 comprises the amino acid sequence of SEQ ID NO: 31; and
the VL CDR3 comprises an amino acid sequence selected from the group consisting of SEQ ID NO: 34, 61 and 62.

10. The antibody or antigen-binding fragment thereof of claim 9, wherein the VH comprises the amino acid sequence of SEQ ID NO: 5 and the VL comprises the amino acid sequence of SEQ ID NO: 6.

11. The antibody or antigen-binding fragment thereof of claim 1, wherein:
the VH CDR1 comprises the amino acid sequence of SEQ ID NO: 67;
the VH CDR2 comprises the amino acid sequence of SEQ ID NO: 17 or 55;
the VH CDR3 comprises the amino acid sequence of SEQ ID NO: 23;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

12. The antibody or antigen-binding fragment thereof of claim 11, wherein:
the VH CDR 1 comprises the amino acid sequence of SEQ ID NO: 12;
the VH CDR2 comprises the amino acid sequence of SEQ ID NO: 17 or 55;
the VH CDR3 comprises the amino acid sequence of SEQ ID NO: 23;
the VL CDR1 comprises the amino acid sequence of SEQ ID NO: 27;
the VL CDR2 comprises the amino acid sequence of SEQ ID NO: 31; and
the VL CDR3 comprises an amino acid sequence selected from the group consisting of SEQ ID NO: 36, 65 and 66.

13. The antibody or antigen-binding fragment thereof of claim 12, wherein the VH comprises the amino acid sequence of SEQ ID NO: 7 and the VL comprises the amino acid sequence of SEQ ID NO: 8.

14. The antibody or antigen-binding fragment thereof of claim 1, wherein:
the VH CDR1 comprises the amino acid sequence of SEQ ID NO: 67;
the VH CDR2 comprises the amino acid sequence of SEQ ID NO: 17 or 55;
the VH CDR3 comprises the amino acid sequence of SEQ ID NO: 25;
the VL CDR1 comprises the amino acid sequence of SEQ ID NO: 68;
the VL CDR2 comprises the amino acid sequence of SEQ ID NO: 31; and
the VL CDR3 comprises the amino acid sequence of SEQ ID NO: 69.

15. The antibody or antigen-binding fragment thereof of claim 14, wherein:
the VH CDR1 comprises the amino acid sequence of SEQ ID NO: 12;
the VH CDR2 comprises the amino acid sequence of SEQ ID NO: 17 or 55;
the VH CDR3 comprises the amino acid sequence of SEQ ID NO: 25;
the VL CDR1 comprises the amino acid sequence of SEQ ID NO: 27;
the VL CDR2 comprises the amino acid sequence of SEQ ID NO: 31; and
the VL CDR3 comprises an amino acid sequence selected from the group consisting of SEQ ID NO: 34, 61 and 62.

16. The antibody or antigen-binding fragment thereof of claim 15, wherein the VH comprises the amino acid sequence of SEQ ID NO: 9 and the VL comprises the amino acid sequence of SEQ ID NO: 10.

17. (canceled)

18. A multispecific antibody comprising an antigen-binding fragment of claim 1 and one or more antibody or antigen-binding fragment having binding specificity to a target antigen that is not FAPα.

19. A chimeric antigen receptor (CAR) comprising an antigen-binding fragment of claim 1, a transmembrane domain, a costimulatory domain, and a CD3ξ intracellular domain.

20. One or more polynucleotide(s) encoding the antibody or antigen-binding fragment thereof of claim 1.

21-24. (canceled)

25. A method of treating cancer or an inflammatory condition in a patient in need thereof, comprising administering to the patient an effective amount of the antibody or antigen-binding fragment thereof of claim 1.

26-29. (canceled)


SEQUENCE LISTING (69 sequences)

SEQ ID NO: 1 - protein, 119 aa, synthetic construct
QVQLLESGGGVVQPGRSLRLSCAASGFTFSSYAMHWVRQAPGKGLEWVAFISHDGNRNSYADSVRGRFTISRDNSKNTLYLQMNSLRAEDTAVYYCARDLGYYNGFSYWGQGTLVTVSA

SEQ ID NO: 2 - protein, 107 aa, synthetic construct
DIVMTQSPSSVSASVGDRVTITCRASQGISSWLAWYQQKPGKAPKLLIYAASSLQSGVPSRFSGSGSGTDFTLTISSLQPEDFATYYCQQANSFPPTFGPGTKVDIK

SEQ ID NO: 3 - protein, 117 aa, synthetic construct
EVQLLESGGDLVQPGGSLRLSCAASGFTFSSYAMSWVRQAPGKGLEWVSGISNGGDSIAYADSVKGRFTVFRDNAKNTLYLQMNSLRAEDTAIYYCARGNYYAHDDWGQGTLVTVSA

SEQ ID NO: 4 - protein, 107 aa, synthetic construct
DIQLTQSPSSVSASVGDRVTITCRASQGVGSWLAWYQQKPGKPPKLLIYAASSLQSGVPSRFSGSGSGTDFTLTISSLQPEDFATYYCQQANSFPLTFGGGTKVEIK

SEQ ID NO: 5 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLSGGTSFDYWGQGTLVTVSA

SEQ ID NO: 6 - protein, 107 aa, synthetic construct
AIRLTQSPSSVSASVGDRVTITCRASQGISSWLAWYQQKPGKAPKLLIYAASSLQSGVPSRFSGSGSGTDFTLTISSLQPEDFATYYCQQANSFPPTFGGGTKVEIK

SEQ ID NO: 7 - protein, 119 aa, synthetic construct
QLQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARDKPWGGSFDYWGQGTLVTVSA

SEQ ID NO: 8 - protein, 107 aa, synthetic construct
DIQLTQSPSSLSASVGDRVTITCRASQGISSWLAWYQQKPGKAPKLLIYAASSLQSGVPSRFSGSGSGTDFTLTISSLQPEDFATYYCQQANSFPVTFGPGTKVDIK

SEQ ID NO: 9 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRTDDTAVYYCARDLTTSTGWESWGQGTLVTVSA

SEQ ID NO: 10 - protein, 107 aa, synthetic construct
NIQMTQSPSSVSASVGDRVTITCRASQGISSWLAWYQQKPGKAPKLLIYAASSLQSGVPSRFSGSGSGTDFTLTISSLQPEDFATYYCQQANSFPPTFGQGTKLEIK

SEQ ID NO: 11 - protein, 5 aa, synthetic construct
SYAMH

SEQ ID NO: 12 - protein, 5 aa, synthetic construct
SYAMS

SEQ ID NO: 13 - protein, 5 aa, synthetic construct
SYGLS

SEQ ID NO: 14 - protein, 5 aa, synthetic construct
SYGMH

SEQ ID NO: 15 - protein, 17 aa, synthetic construct
FISHDGNRNSYADSVRG

SEQ ID NO: 16 - protein, 17 aa, synthetic construct
GISNGGDSIAYADSVKG

SEQ ID NO: 17 - protein, 17 aa, synthetic construct
AISGSGGSTYYADSVKG

SEQ ID NO: 18 - protein, 17 aa, synthetic construct
WISPYTPNPIYAPNFRD

SEQ ID NO: 19 - protein, 17 aa, synthetic construct
VISYDGSNKYYADSVKG

SEQ ID NO: 20 - protein, 10 aa, synthetic construct
DLGYYNGFSY

SEQ ID NO: 21 - protein, 8 aa, synthetic construct
GNYYAHDD

SEQ ID NO: 22 - protein, 10 aa, synthetic construct
DLSGGTSFDY

SEQ ID NO: 23 - protein, 10 aa, synthetic construct
DKPWGGSFDY

SEQ ID NO: 24 - protein, 13 aa, synthetic construct
NHWVGRPTRAFDL

SEQ ID NO: 25 - protein, 10 aa, synthetic construct
DLTTSTGWES

SEQ ID NO: 26 - protein, 11 aa, synthetic construct
ESYTGLGSFLH

SEQ ID NO: 27 - protein, 11 aa, synthetic construct
RASQGISSWLA

SEQ ID NO: 28 - protein, 11 aa, synthetic construct
RASQGVGSWLA

SEQ ID NO: 29 - protein, 12 aa, synthetic construct
RASQTVSITHLA

SEQ ID NO: 30 - protein, 11 aa, synthetic construct
RASQTISNYLN

SEQ ID NO: 31 - protein, 7 aa, synthetic construct
AASSLQS

SEQ ID NO: 32 - protein, 7 aa, synthetic construct
GASARAT

SEQ ID NO: 33 - protein, 7 aa, synthetic construct
AASSLQG

SEQ ID NO: 34 - protein, 9 aa, synthetic construct
QQANSFPPT

SEQ ID NO: 35 - protein, 9 aa, synthetic construct
QQANSFPLT

SEQ ID NO: 36 - protein, 9 aa, synthetic construct
QQANSFPVT

SEQ ID NO: 37 - protein, 8 aa, synthetic construct
QHYGSSIT

SEQ ID NO: 38 - protein, 5 aa, synthetic construct
QYWGT

SEQ ID NO: 39 - protein, 122 aa, synthetic construct
EVQLVQSGAEVKEPGVSVKVSCKASGYTFASYGLSWVRLAPGQGLQWMGWISPYTPNPIYAPNFRDKVILTTDRSTNTAYLEVRSLRSDDTGLYYCARNHWVGRPTRAFDLWGQGTMVTVSA

SEQ ID NO: 40 - protein, 107 aa, synthetic construct
EIVLTQSPGTLSLSPGERATLSCRASQTVSITHLAWYQQKPGQAPRLLTYGASARATGIPDRFSVSGSGTDFTLTITRLEPEDFAVYYCQHYGSSITFGGGTKVEIK

SEQ ID NO: 41 - protein, 120 aa, synthetic construct
QVQLLESGGGVVQPGRSLRLSCAASGFTFSSYGMHWVRQAPGKGLEWVAVISYDGSNKYYADSVKGRFTISRDNSKNTLYLQMNSLRADDTAVYYCARESYTGLGSFLHWGQGTLVTVSA

SEQ ID NO: 42 - protein, 103 aa, synthetic construct
NIQLTQSPSSLSASVGDRVTITCRASQTISNYLNWYQQKPGKAPKLLISAASSLQGGVPSRFSGSGSGTDFTLAISSLQPEDSATYYCQYWGTFGQGTKLEIK

SEQ ID NO: 43 - protein, 17 aa, synthetic construct
FISHDANRNSYADSVRG

SEQ ID NO: 44 - protein, 17 aa, synthetic construct
FISHDGNRNAYADSVRG

SEQ ID NO: 45 - protein, 17 aa, synthetic construct
FISHDGNRQSYADSVRG

SEQ ID NO: 46 - protein, 17 aa, synthetic construct
FISHDGNRNSYADAVRG

SEQ ID NO: 47 - protein, 17 aa, synthetic construct
FISHDANRNAYADAVRG

SEQ ID NO: 48 - protein, 17 aa, synthetic construct
FISHDANRQSYADAVRG

SEQ ID NO: 49 - protein, 17 aa, synthetic construct
GISNAGDSIAYADSVKG

SEQ ID NO: 50 - protein, 17 aa, synthetic construct
GISQGGDSIAYADSVKG

SEQ ID NO: 51 - protein, 17 aa, synthetic construct
GISNGGDAIAYADSVKG

SEQ ID NO: 52 - protein, 17 aa, synthetic construct
GISNGGDSIAYADAVKG

SEQ ID NO: 53 - protein, 17 aa, synthetic construct
GISNAGDAIAYADAVKG

SEQ ID NO: 54 - protein, 17 aa, synthetic construct
GISQGGDAIAYADAVKG

SEQ ID NO: 55 - protein, 17 aa, synthetic construct
AISGSGGSTYYADAVKG

SEQ ID NO: 56 - protein, 17 aa, synthetic construct
VISYDASNKYYADSVKG

SEQ ID NO: 57 - protein, 17 aa, synthetic construct
VISYDGSNKYYADAVKG

SEQ ID NO: 58 - protein, 17 aa, synthetic construct
VISYDASNKYYADAVKG

SEQ ID NO: 59 - protein, 10 aa, synthetic construct
DLGYYNAFSY

SEQ ID NO: 60 - protein, 10 aa, synthetic construct
DLGYYQGFSY

SEQ ID NO: 61 - protein, 9 aa, synthetic construct
QQANAFPPT

SEQ ID NO: 62 - protein, 9 aa, synthetic construct
QQAQSFPPT

SEQ ID NO: 63 - protein, 9 aa, synthetic construct
QQANAFPLT

SEQ ID NO: 64 - protein, 9 aa, synthetic construct
QQAQSFPLT

SEQ ID NO: 65 - protein, 9 aa, synthetic construct
QQANAFPVT

SEQ ID NO: 66 - protein, 9 aa, synthetic construct
QQAQSFPVT

SEQ ID NO: 67 - protein, 5 aa, synthetic construct
  VARIANT 5: note = X is H or S
SYAMX

SEQ ID NO: 68 - protein, 11 aa, synthetic construct
  VARIANT 6: note = X is I or V
  VARIANT 7: note = X is G or S
RASQGXXSWLA

SEQ ID NO: 69 - protein, 9 aa, synthetic construct
  VARIANT 4: note = X is N or W
  VARIANT 5: note = X is A or S
  VARIANT 8: note = X is L, P or V
QQAXXFPXT